(19) United States
(12) Patent Application Publication
(10) Pub. No.: US 2015/0174236 A1
(43) Pub. Date: Jun. 25, 2015

(54) VIRAL VACCINE AND PROCESS FOR PREPARING THE SAME

(71) Applicant: GEORGE DACAI LIU, REDMOND, WA (US)

(72) Inventor: GEORGE DACAI LIU, REDMOND, WA (US)

(21) Appl. No.: 14/642,934

(22) Filed: Mar. 10, 2015

Related U.S. Application Data
(63) Continuation-in-part of application No. 13/279,252, filed on Oct. 22, 2011.

(51) Int. Cl. A61K 39/21; G01N 33/543; A61K 39/135; C12N 7/00; A61K 39/145; A61K 39/12

(52) U.S. Cl. CPC A61K 39/21; A61K 39/145; A61K 39/12; A61K 39/135; C12N 7/00; G01N 33/54306; A61K 2039/525

(57) ABSTRACT

The present invention provides a vaccine against a viral infection. The exemplary vaccine comprises a viral antigen of a vaccine strain of a virus; wherein the viral antigen is derived from a virus preparation of the vaccine strain of the virus; wherein the virus preparation of the vaccine strain of the virus contains a subpopulation of infectious viral particles, and the subpopulation of infectious viral particles is represented as a proportion over the total viral particles or total viral antigens of the virus preparation; and wherein the proportion of the subpopulation of infectious viral particles over the total viral particles or total viral antigens of the virus preparation is over a predefined threshold; so that the vaccine provides at least partial inter-subtypic or intra-subtypic cross immune response against different strains of the virus than the vaccine strain.

### CROSS REFERENCE TO RELATED APPLICATIONS

The present application claims the priority of U.S. provisional application No. 61/408,575 filed on Oct. 30, 2010, entitled of “Virus vaccines and compositions and processes for preparing the same”, and U.S. application Ser. No. 13/279,252 filed on Oct. 22, 2011, the disclosure of them is herein incorporated in their entirety.

### FIELD OF THE INVENTION

The present invention relates to viral vaccines comprising a viral antigen derived from a virus preparation with an enriched subpopulation of infectious particles and further to processes for making the same.

### BACKGROUND OF THE INVENTION

Dreadfully infectious viruses such as human immunodeficiency virus (HIV), influenza virus, dengue fever virus (DENV), and foot and mouth disease virus (FMDV) are still causing grave consequences in humans and animals. Vaccines are considered as the most effective and economic means for prevention from and therapy of viral infections. Unfortunately, the viruses like HIV, influenza virus, DENV, FMDV are comprised of many serotypes (i.e., subtypes), and undergo rapid antigenic changes; these make it a grave challenge to produce an effective vaccine for inter-subtypic and/or intra-subtypic cross protections.

Influenza A viruses are responsible for the major pandemics of influenza in the last century and also the causative agents for most of the annual outbreaks of epidemic influenza. The WHO estimates that epidemic influenza affects approximately 5-15% of the global population annually, and is responsible for up to 3-5 million cases of severe disease and 500,000 deaths per year. WHO Influenza Fact sheet 211. World Health Organisation, Geneva, Switzerland (2003).

Influenza A virus is a member of the Orthomyxovirus family, and has a wide host range, including humans, horses, dogs, birds, and pigs. It is an enveloped, negative-sense RNA virus composed of a set of 8 RNA segments (abbreviated as PB2, PB1, PA, HA, NP, NA, M and NS) encoding at least 10 viral proteins. The HA segment encodes the hemagglutinin (HA) protein. The NA segment encodes the neuraminidase (NA). Based on serological classification, 16 HA subtypes (designated as H1 through H16) and 9 NA subtypes (designated as N1 through N9) have been thus far identified. Subtypes of influenza A that are currently circulating among people worldwide include H1N1, H1N2, and H3N2 viruses; H5N1 and H9N2 are circulating in birds such chickens; and H1N1 and H3N2 are circulating in pigs.

Current inactivated influenza vaccines are trivalent, containing 15 μg HA of two influenza A (H1N1 and H3N2) subtypes and one influenza B strain. The basic technology and principles of vaccine production have remained much the same since their first introduction into clinical uses in the 1940s. The conventional wisdoms have focused on the optimization of production procedures to produce a conventional virus preparation with the maximum amount of HA proteins. In addition, influenza vaccines are standardized solely on the basis of HA content.

Vaccine efficacy declines as the antigenic relatedness between the circulating viruses and the viruses selected for the vaccine becomes more distant within the same subtype. For influenza vaccines, strain selection of the three viruses to be included in the annual seasonal vaccine now occurs twice a year at the WHO. While the selected strains are usually antigenically close to circulating strains, in some years they are not. Therefore, there is a need to have vaccines that will produce broadened protective immunity.

For HIV-1, there are already 33 million infected individuals who each harbors a substantial array of HIV-1 quasi-species, which results in an enormous number of variants that are simultaneously seeded and circulating in the human population. Providing protection against this vast array of potentially infectious isolates is a challenge of unprecedented magnitude in vaccine development. Not surprisingly, the conventional vaccine approaches of chemical inactivation or live attenuation have not produced a broadly protective or safe HIV-1 vaccine.

FMDV infects food producing animals such as cattle, sheep, goats, and swine. FMDV a non-enveloped virus with icosahedral symmetry and approximately 30 nm diameter is extremely labile in vitro. The efficacy of inactivated virus vaccines is highly dependent on virus integrity. The 140S quantitative sucrose density gradient analysis is the recommended method to quantify virus antigen and, on that basis, formulate vaccines. However, FMDV vaccines formulated solely on the basis of 140S amount fail frequently in the field.

Dengue virus (DENV) with four serotypes is the cause of dengue fever. It is a single positive-stranded RNA virus of the family Flaviviridae. Its genome is about 11000 bases that codes for three structural proteins, capsid protein C, membrane protein M, envelope protein E; seven nonstructural proteins, NS1, NS2a, NS2b, NS3, NS4a, NS4b, NS5. The rate of nucleotide substitution for this virus has been to be 6.5×10⁻⁴ per nucleotide per year, a rate similar to other RNA viruses. No vaccine is currently available.

### SUMMARY OF THE INVENTION

One object of the present invention is to provide a vaccine that is able to illicit at least partial inter-subtypic or intra-subtypic cross immunity against a virus.

One aspect of the present invention provides a vaccine. In one embodiment, the vaccine comprises a viral antigen derived from a virus preparation of a vaccine strain of a virus; wherein the virus preparation contains a subpopulation of infectious viral particles, and total viral particles (TVPs) or total viral antigens (TVAs); and the infectious viral particles and TVPs or TVAs are over predetermined values.

In another embodiment of the vaccine, when the infectious viral particles are measured as plaque-forming units (PFU) and the TVPS are measured by viral particle numbers, the virus preparation has the following characteristics: PFU is over 1×10⁷/ml, or 2×10⁷/ml, or 3×10⁷/ml, or 4×10⁷/ml, or 5×10⁷/ml, or 6×10⁷/ml, or 7×10⁷/ml, or 8×10⁷/ml; TVPs are over 1×10⁸/ml, or 2×10⁸/ml, or 3×10⁸/ml, or 4×10⁸/ml; and ratio of PFU/TVPs is over 2%, or 3%, or 4%, or 5%, or 6%, or 7%, or 8%, or 9%, or 10%, or 11%, or 12% or 13%.

In another embodiment of the vaccine, wherein when the infectious viral particles are measured as PFU and the TVAs are measured as a numeric value, the virus preparation has the following characteristics: PFU is over 1×10⁷/ml, or 2×10⁷/ml, or 3×10⁷/ml, or 4×10⁷/ml, or 5×10⁷/ml, or 6×10⁷/ml, or 7×10⁷/ml, or 8×10⁷/ml; and TVA is about 10-60% of the highest TVA value in a panel of virus preparations containing the virus preparation.

In another embodiment of the vaccine, when the infectious viral particles are measured as TCID₅₀ and the TVAs are measured as a numeric value, the virus preparation has the following characteristics: TCID50 is over 1×10⁸/ml, or 2×10⁸/ml, or 3×10⁸/ml, or 4×10⁸/ml, or 5×10⁸/ml, or 6×10⁸/ml, or 7×10⁸/ml, or 8×10⁸/ml; and TVA is about 10-60% of the highest TVA value in a panel of virus preparations containing the virus preparation.

In another embodiment of the vaccine, the virus is able to infect humans and animals. In a further embodiment of the vaccine, the virus is influenza virus, HIV, DENV or FMDV.

In another embodiment of the vaccine, the viral antigen is a surface antigen of the virus.

Another aspect of the present invention provides a method for producing a vaccine. In one embodiment, the method comprises providing a virus preparation of a vaccine strain of a virus; wherein the virus preparation contains a subpopulation of infectious viral particles, and total viral particles (TVPs) or total viral antigens (TVAs), wherein the infectious viral particles and the TVPs or TVAs are over predetermined values; deriving a viral antigen from the virus preparation; and mixing the viral antigen with a physiologically acceptable adjuvant to make the vaccine.

In another embodiment of the method, the predetermined values are determined by providing a panel of virus preparations; measuring the infectious viral particles and TVPs or TVAs of each of the panel of virus preparations; preparing vaccines with the viral antigens derived from the panel of virus preparations; immunizing a suitable host with the prepared vaccines; measuring the cross immune responses of each of the prepared vaccines; and analyzing the cross immune responses; wherein the values of the infectious viral particles and TVPs or TVAs from a virus preparation that provides desired cross immune responses are determined as the predetermined values. In a further embodiment of the method, the panel of virus preparations are obtained by diluting a virus stock of the vaccine strain into a plurality of dilutions as working stocks; infecting cells with the working stocks and incubating the infected cells; and collecting viruses at different incubating time points to obtain the panel of virus preparations.

### DETAILED DESCRIPTION OF THE INVENTION

The present invention may be understood more readily by reference to the following detailed description of certain embodiments of the invention.

Throughout this application, where publications are referenced, the disclosures of these publications are hereby incorporated by reference, in their entireties, into this application in order to more fully describe the state of art to which this invention pertains.

The practice of the present invention will employ, unless otherwise indicated, conventional techniques of molecular biology (including recombinant techniques), microbiology, cell biology, biochemistry, nucleic acid chemistry, and immunology, which are within the skill of the art. Such techniques are explained fully in the literature, such as, Molecular Cloning: A Laboratory Mannual, third edition (Sambrook and Russel, 2001); Animal Cell Culture (R. I. Freshmey, ed., 1987); Current Protocols in Molecular Biology (F. M. Ausubel et al., eds., 1987, including supplements through 2001); Current Protocols in Immunology (J. E. Coligan et al., eds., 1991); The Immunoassay Handbook (D. Wild, ed., Stockton Press NY, 1994); Methods of Immunological Analysis (R. Masseyeff, W. H. Albert, and N. A. Staines, eds., Weinheim: VCH Verlags gesellschaft mbH, 1993).

Viruses replicate inside cells; thus virus preparations can be produced by propagating viruses in for example chicken embryos, ex vivo tissues, and cultured cells. For example, influenza viruses are propagated in chicken embryos and Vero cells for producing influenza vaccines; FMDV is propagated in BHK-21 cells for producing FMDV vaccines. Under the current dogma of vaccine productions, influenza virus preparations used for vaccines are optimized based on their HA titers in hemagglutination assays, and FMDV preparations used for vaccines on their TCID₅₀ titers. However, the HA titers represents the amount of all viral particle subpopulations without regard to the proportion of any particular subpopulation, and the TCID₅₀ titer represents only the subpopulation of infectious viral particles without regard to its proportion in the amount of all viral particle subpopulations. It is apparent that a virus preparation used for vaccine under current understanding and practice of vaccine industry is usually measured by one parameter, let alone two parameters or relations between two parameters.

Throughout this application, a “virus preparation” refers to a virus stock that will be used for vaccine production; in certain embodiments, the virus preparation can be the one prepared by propagating viruses in for example chicken embryos, ex vivo tissues, and cultured cells without being further manipulated; in certain embodiments, the virus preparation can be the one that has been purified, concentrated, enriched or separated before the viral antigens are used for vaccine production. Since measuring the infectious viral particles needs live viral particles, the virus preparation shall not be inactivated before such measurement is done. If any parameter is experimentally maintained under manipulation such as purification, concentration, enrichment or separation, the parameter can be measured before such manipulation.

It is well known that a virus preparation contains all viral particle subpopulations, for example infectious viral particle subpopulation and non-infectious viral particle subpopulation. In the pursuit of producing a viral vaccine with broadened immunity against inter-subtypic or intra-typic strains, the inventor of the present invention discovered that the proportion of the subpopulation of infectious viral particles in a virus preparation was critical for producing the vaccine that could elicit broadened immunity, and further that the proportion of the subpopulation of infectious viral particles in a virus preparation could be increased by for example optimized culture conditions. The present invention offers at least partial explanation of why the current influenza and FMDV vaccines fail to elicit intra-sutypic or inter-subtypic cross immunity and why no HIV or Dengue vaccine is available. More importantly, the present invention provides principles for producing a viral vaccine that is capable of eliciting broadened intra-subtypic and inter-subtypic cross immunity against any virus infection.

The present invention provides the viral vaccines comprising a viral antigen derived from a virus preparation with defined proportions of subpopulations of infectious viral particles and total viral particles or total viral antigens. The present invention further provides methods for making such vaccines.

During virus propagation, both infectious and non-infectious viral particles are generated, where the infectious particles are defined as the particles that can for example form plaques in a cell-based plaque colony-forming assay (i.e., plaque-forming particles) or cause CPE or cause clinical symptoms when administrated into a host, and the non-infectious particles as the particles that cannot perform the infectious functions of the infectious particles. In addition, many viral fragments and soluble antigens are also produced during virus propagation. In the simplest application of the principles of the present invention, two parameters of a virus preparation are measured, one for the subpopulation of infectious viral particles (expressed for example as plaque-forming units (PFU), TCID50, EID50, LD50), and the other for the total viral particles (expressed for example as HA titers) or the total viral antigens (expressed for example as ELISA readings, Western blot intensities); then the proportion of the subpopulation of infectious viral particles in the virus preparation is calculated as a ratio between the subpopulation of infectious viral particles and the subpopulation of total viral particles or total viral antigens. If a virus preparation can induce desirable cross-protection, the ratio is deemed optimal.

A virus preparation with an optimal ratio can be determined by experiments without undue experimentation. For example, the optimal ratio of a virus preparation from an influenza virus strain (H3N2) can be determined as follows: a panel of virus preparation are prepared by a combination of different inoculation doses (e.g., different dilutions of the same stock, covering all practically possible dilutions) and different incubation time periods (e.g., 24, 36, 48, 72 hours post-inoculation, covering all practically possible time points), then the infectious and total viral particles (TVPs) in each virus preparation are measured by PFU and HA titers; then a few of the virus preparations are selected for animal experiments; the optimal ratio is determined by the virus preparation that provides the desired cross protection or antibody titers. For a virus of which the infectious (as PFU) and total viral particles (TVPs) can be measured by particle numbers, the virus preparations that can be used for vaccine production have the following characteristics: PFU is over 1×10⁷/ml, or 2×10⁷/ml, or 3×10⁷/ml, or 4×10⁷/ml, or 5×10⁷/ml, or 6×10⁷/ml, or 7×10⁷/ml, or 8×10⁷/ml; TVPs are over 1×10⁸/ml, or 2×10⁸/ml, or 3×10⁸/ml, or 4×10⁸/ml; and the ratio of PFU/TVPs is over 2%, or 3%, or 4%, or 5%, or 6%, or 7%, or 8%, or 9%, or 10%, or 11%, or 12% or 13%. For FMDV, the virus preparations can be prepared following the same procedure; since no current method was available for measuring the TVPs, the total viral antigens (TVA) are measured by ELISA; in addition, the infectious viral particles are measured by both PFU and TCID50; thus, for virus preparations derived from FMDV or the like, the virus preparations that can be used for vaccine production have the following characteristics: 1) when PFU and TVA are measured, PFU is over 1×10⁷/ml, or 2×10⁷/ml, or 3×10⁷/ml, or 4×10⁷/ml, or 5×10⁷/ml, or 6×10⁷/ml, or 7×10⁷/ml, or 8×10⁷/ml; TVA is about 10-60% of the highest TVA value in the panel of virus preparation, preferably about 20-60% of the highest TVA value in the panel of virus preparation, more preferably about 30-50% of the highest TVA value in the panel of virus preparation; 2) when TCID50 and TVA are measured, TCID50 is over 1×10⁸/ml, or 2×10⁸/ml, or 3×10⁸/ml, or 4×10⁸/ml, or 5×10⁸/ml, or 6×10⁸/ml, or 7×10⁸/ml, or 8×10⁸/ml; TVA is about 10-60% of the highest TVA value in the panel of virus preparation, preferably about 20-60% of the highest TVA value in the panel of virus preparation, more preferably about 30-50% of the highest TVA value in the panel of virus preparation. Of course, the infectious viral particles can be measured by other methods such as EID50, LD50, and the total viral particles/antigens can be measured by any other suitable methods. In certain embodiments, a selected virus preparation after propagation is subject to physical separation (e.g., gradient centrifuge) so as to obtain a fraction of the viruses that contains higher population of infectious particles. It is to be noted that the physical separation can be done after the inactivation of the viruses in order to minimize the biohazard when preliminary studies have identified the portion that would contain the higher population of infectious particles if the viral stock is separated with live viruses. In certain embodiments, the virus preparation containing optimal population of infectious particles resulting from optimal propagation is subject to further physical separation so that the population of infectious particles is further increased or enriched. Apparently, any process that can isolate infectious particles in, to a certain extent, purity is suitable for the present invention.

The enrichment of infectious particles in the viral antigens used for vaccines and pharmaceutical compositions of the present invention enhances the induction of cross-protection immune responses. Without wish to be bound by any particular theories, the present invention reasoned that 1) viral particles during viral production are comprised of infectious particles and non-infectious ones, where the infectious particles must contain the surface antigens that bear proper receptor-binding epitopes for successful infection while the non-infectious particles might be deficient in such antigens; while no data of infectious particles in any viral preparation for current vaccine manufactures is available, our experiments showed that the viral stock of influenza virus resulting from common propagation conditions (i.e., inoculation doses with high dilutions (100-10000 dilution) and long incubation period (48-72 hours)) had low percentage (less than 1%) of infectious particles in the total viral particles; 2) the cell surface receptors for one specific virus do not mutate at all or very rarely, implying that different subtypes or serotypes of the specific virus for example influenza virus shall have the same or substantially similar receptor-binding epitope or domain in their receptor binding molecules (i.e., surface antigens); the proper receptor-binding epitopes on the surface antigens are more likely to be shared among the infectious particles of different subtypes or serotypes within one specific virus. Therefore, the lack or ineffectiveness of cross-protection of current vaccines may be due to insufficient antibodies specific for receptor-binding epitope or domain because the receptor-binding epitope or domain present in the small percentage of infectious particles in the total viral antigens might be overlooked by the host immune system. Furthermore, if the percentage of infectious particles is increased, it expects to increase the presence of the proper receptor-binding epitope or domain in the total viral antigens; and when the number of the proper receptor-binding epitope or domain reaches a point where enough antibody responses specific for the receptor-binding epitope or domain are elicited so as to react with different subtypes or serotypes, resulting in cross protection.

The methods for increasing the contents of infectious particles in a viral preparation include any suitable ones. For example, the suitable viral preparation can be selected by inoculating embryonated eggs or cells with different dilutions of seed viruses and then incubating for different time periods, and then assaying for their total viral antigens (e.g., for influenza A viruses, HA assay or HA protein contents) and infectious particles (e.g., for influenza A viruses, cell-based plaque assay) and then determining the conditions by which suitable viral preparations can be produced. In addition, it is reasonable to believe that the infectious particles are physically different from other particles or incomplete particles or soluble antigens; thus it is possible to separate the infectious particles from the rest of the viral preparation to obtain a viral preparation with higher contents of infectious particles for vaccines. The ideal situation is that the infectious particles can be specifically separated from the rest of the viral preparation. For example, the density of infectious particles may be unique, so that gradient ultra-centrifugation may be used to obtain the fractions with enriched infectious particles. It is evident that the optimal viral preparations can be obtained by combining two or more methods.

As used herein, a “vaccine” is an antigenic preparation that is used to induce an immune response in individuals. A vaccine can have more than one constituent that is antigenic.

As used herein, “non-protein carriers” are carriers which are not proteins and can be used to achieve multimeric display of influenza matrix and/or nucleoprotein.

“Adjuvant” refers to a substance which, when added to an immunogenic agent such as antigen, nonspecifically enhances or potentiates an immune response to the agent in the recipient individual upon exposure to the mixture.

The term “microcarrier” refers to a particulate composition which is insoluble in water and which has a size of less than about 150, 120 or 100 um, more commonly less than about 50-60 um, and may be less than about 10 um or even less than about 5 um. Microcarriers include “nanocarriers,” which are microcarriers have a size of less than about 1 um, preferably less than about 500 nm. Microcarriers include solid phase particles such particles formed from biocompatible naturally occurring polymers, synthetic polymers or synthetic copolymers, although microcarriers formed from agarose or cross-linked agarose may be included or excluded from the definition of microcarriers herein as well as other biodegradable materials known in the art.

An “individual” or “subject” is a vertebrate, such as avian, preferably a mammal, such as a human. Mammals include, but are not limited to, humans, non-human primates, farm animals, sport animals, experimental animals, rodents (e.g., mice and rats) and pets.

An “effective amount” or a “sufficient amount” of a substance is that amount sufficient to effect a desired biological effect, such as beneficial results, including clinical results, and as such, an “effective amount” depends upon the context in which it is being applied. In the context of this invention, an example of an effective amount of a composition comprising the desired antigen is an amount sufficient to induce an immune response in an individual. An effective amount can be administered in one or more administrations.

“Stimulation” of an immune response, such as humoral or cellular immune response, means an increase in the response, which can arise from eliciting and/or enhancement of a response.

As used herein, and as well-understood in the art, “treatment” is an approach for obtaining beneficial or desired results, including clinical results. For purposes of this invention, beneficial or desired clinical results include, but are not limited to, alleviation or amelioration of one or more symptoms, diminishment of extent of infection, stabilized (i.e., not worseing) state of infection, amelioration or palliation of the infectious state, and remission (whether partial or total), whether detectable or undetectable. “Treatment” can also mean prolonging survival as compared to expected survival if not receiving treatment.

According to the present invention, a “dose” of a vaccine composition, is a quantity of vaccine composition that is administered at a particular point in time. A “dose” may also be a quantity of vaccine composition that is gradually administered to an animal using an extended release formulation and/or apparatus. In certain embodiments of the present invention, two or more doses of the vaccine composition are administered to an animal at different time points.

According to the present invention, an “immunologically-effective amount” of an influenza virus (e.g., an inactivated influenza virus) is an amount of influenza virus (usually expressed in terms of hemagglutinating units or “HA units”) which will induce complete or partial immunity in a treated animal against subsequent challenge with a virulent strain of avian influenza virus. Complete or partial immunity can be assessed by observing, either qualitatively or quantitatively, the clinical symptoms of influenza virus infection in a vaccinated animal as compared to an unvaccinated animal after being challenged with a virulent strain of avian influenza virus. Where the clinical symptoms of influenza virus infection in a vaccinated animal after challenge are reduced, lessened or eliminated as compared to the symptoms observed in an unvaccinated animal after a similar or identical challenge, the amount of influenza virus that was administered to the vaccinated animal is regarded as an “immunologically-effective amount”.

A “cross-protective immune response” is one which protects against infection by a virus strain which is not identical to the one used to elicit the response; the “cross-protective immune response” could be inter-subtypic or intra-subtypic.

Unless defined otherwise, all technical and scientific terms used herein have the same meaning as commonly understood by one of ordinary skill in the art to which this invention belongs.

Methods of determining HA units are known in the art. As used herein, an HA unit is defined as the reciprocal of the highest dilution of an influenza virus-containing sample which causes visible hemagglutination when combined with erythrocytes.

A virus includes, but not limited to, influenza virus, HIVs, FDMV, Dengue fever virus, hepatitis C virus, Ebola virus, measles virus, parainfluenza virus and respiratory syncytial virus or any virus of which the antigenc epitopes are subject to continuous alteration in circulating epidemic strains. Such viruses may include, but are not limited to paramyxoviruses (Sendai Virus, parainfluenza virus, mumps, Newcastle disease virus), morbillivirus (measles virus, canine distemper virus and rinderpest virus); pneumovirus (respiratory syncytial virus and bovine respiratory virus); rhabdovirus (vesicular stomatitis virus and lyssavirus).

Methods for producing influenza virus in cell culture are known in the art. The virus may be grown on cells of mammalian, avian, or human origin, such as Madin Darby Canine Kidney (MDCK), Vero, MDBK, CLDK, Ebx or PerC6 cells. For FMDV, BHK-21 cells are suitable.

The dose of a viral antigen is between 0.1 and 60 g, preferably between 3 and 30 g, and most preferably between 5 and 15 g. The viral antigen can be in the form of inactivated viral particles, split viral antigens, virosomes or purified antigens.

Non-limiting examples of suitable adjuvants include squalane and squalene (or other oils of animal origin); block copolymers; detergents such as Tween-80; Quil A, mineral oils such as Drakeol or Marcol, vegetable oils such peanut oil; Corynebacterium-derived adjuvants such as Corynebacterium parvum; Propionibacterium-derived adjuvants such as Propionibacterium acne; Mycobacterium bovis (Bacille Calmette and Guerin or BCG); interleukins such as interleukin 2 and interleukin 12; monokines such as interleukin 1; tumor necrosis factor, interferons such as gamma interferon; surface active substances such as hexadecylamine, octadecylamine, octadecyl amino acid esters, lysolecithin, dimethyldioctadecylammonium bromide, N,N-dicoctadecyl-N′,N′bis)2-hydroxyethyl-propanediamine), methoxyhexadecylglycerol, and pluronic polyols; polyamines such as pyran, dextransulfate, poly IC carbopol; peptides such as muramyl dipeptide and derivatives, dimethylglycine, tuftsin; oil emulsions; and mineral gels such as aluminum phosphate, aluminum hydroxide or alum; combinations such as saponin-aluminium hydroxide or Quil-A aluminium hydroxide; liposomes; mycobacterial cell wall extract; synthetic glycopeptides such as muramyl dipeptides or other derivatives; Avridine; Lipid A derivatives; dextran sulfate; DEAE-Dextran or with aluminium phosphate; carboxypolymethylene such as Carbopol'EMA; acrylic copolymer emulsions such as Neocryl A640; vaccinia or animal poxvirus proteins; sub-viral particle adjuvants such as cholera toxin, or mixtures thereof.

Mono- or disaccharide derivatives having at least one but no more than N-1 fatty acid ester groups and, optionally, one but not more than N-1 sulphate ester groups, wherein N is the number of hydroxyl groups of the mono- or disaccharide from which the derivative is derived. Sucrose fatty acid sulphate ester incorporated in a submicron squalane-in-water emulsion. The dose of sucrose fatty acid sulphate ester is between 0.1 and 40 mg. Preferably, the dose of sucrose fatty acid sulphate ester is between 0.5 and 10 mg. Most preferably, the dose of sucrose fatty acid sulphate ester is between 0.5 and 4 mg. The dose of squalane is between 0.4 and 160 mg. Preferably, the dose of squalane is between 1 and 40 mg. Most preferably, the dose of squalane is between 2 and 16 mg.

A therapeutic composition of the present invention can be formulated in an excipient that the object to be treated can tolerate. Examples of such excipients include water, saline, Ringer's solution, dextrose solution, Hank's solution, and other aqueous physiologically balanced salt solutions. Excipients can also contain minor amounts of additives, such as substances that enhance isotonicity and chemical or biological stability. Examples of buffers include phosphate buffer, bicarbonate buffer, and Tris buffer, while examples of stabilizers include A1/A2 stabilizer, available from Diamond Animal Health, Des Moines, Iowa.

Individuals, in the context of this application, refer to birds and/or mammals such as, but not limited to, apes, chimpanzees, orangutans, humans, monkeys or domesticated animals (pets) such as dogs, cats, guinea pigs, hamsters, rabbits, ferrets, cows, horses, goats and sheep. Avian or bird is herein defined as any warm-blooded vertebrate member of the class Aves typically having forelimbs modified into wings, scaly legs, a beak, and bearing young in hard-shelled eggs. For purposes of this specification, preferred groups of birds are domesticated chickens, turkeys, ostriches, ducks, geese, swan, and cornish game hens. A more preferred group is domesticated chickens and turkeys.

Acceptable protocol to administer therapeutic compositions in an effective manner includes individual dose size, number of doses, frequency of dose administration, and mode of administration. Determination of such protocols can be accompanished by those skilled in the art, and examples are disclosed herein.

Administering or administer is defined as the introduction of a substance into the body of an individual and includes oral, nasal, ocular, rectal, vaginal and parenteral routes. Compositions may be administered individually or in combination with other agents via any route of administration, including but not limited to subcutaneous (SQ), intramuscular (IM), intravenous (IV), intraperitoneal (IP), intradermal (ID), via the nasal, ocular or oral mucosa (IN) or orally.

The dose administered to a patient, in the context of the present invention, should be sufficient to effect a beneficial response in a patient over an appropriate period of time. The quantity of agents to be administered may depend on the subject to be treated inclusive of the age, sex, weight and general health condition thereof, factors that will depend on the judgment of the practitioner.

Immunotherapeutic compositions of the invention may be used to prophylactically or therapeutically immunize animals such as humans. However, other animals are contemplated, preferably vertebrate animals including domestic animals such as livestock and companion animals.

The vaccine may be used in combination with others; for example, priming with an attenuated vaccine follows with a boost using the inactivated vaccine.

The invention encompasses all pharmaceutical compositions comprising an antigen, an adjuvant, and a pharmaceutically acceptable carrier.

Pharmaceutically acceptable carriers preferred for use in the present invention may include sterile aqueous of non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose”, and the like. Preservatives and other additives may also be present such as, for example, antimicrobials, antioxidants, chelating agents, and inert gases and the like.

### EXAMPLES

The following examples are provided for the sole purpose of illustrating the principles of the present invention; they are by no means intended as limitations of the present invention.

I. Influenza Virus

1. Virus Strains

Influenza A/swine/Guangdong/01/2002 (H3N2) was isolated from a healthy pig in south China.

2. Virus Propagation in Chicken Embryos

Ten-day old embryonated eggs were used for virus replication. The eggs were inoculated with 0.2 ml of the virus inoculum with different dilutions from a viral stock. The inoculated eggs were incubated at the appropriate temperature for indicated time periods. At the end of the incubation period, the embryos were killed by cooling and the eggs were stored for 12-60 hours at 2-8° C. The allantoic fluids from the chilled embryonated eggs were harvested.

3. HA Assay and Plaque Forming Assay

The HA assay and plaque forming assay were well known in the art. Chicken red blood cells were used for the HA assay. MDCK cells were used for the plaque forming assay. The results are summarized in Table 1. PFU was considered as an indicator of infectious particles, where HA titer was considered as an indicator of total hemagglutinin proteins. For calculation, one HA unit is equal to 5×10⁶ viral particles (hemagglutinating particles (HAP)). In current influenza vaccines the content of hemagglutinin is the sole indicator used for determining the doses of vaccines. Here the ratio of PFU over HA titer (HAP) was used for selecting the viral stocks for testing their efficacy as vaccines. The group of 10⁻⁶ dilution with 36 hour incubation gave rise to the second best ratio (but at 24 hours, even though the ratio is the highest, it had very low HA titer, the total antigens were too low for practice use), whereas the group of 10⁻² dilution with 72 hour incubation was the worst (but it had the highest HA; it would be optimal for current procedures for making influenza vaccine); thus these two groups were used for further experiments.

TABLE 1

| | Incubation | | | |
|---|---|---|---|---|
| Dilution | time | PFU | HA (HAP) | PFU/HAP |
| 10⁻² | 24 hours | 5.73 × 10⁷ | 2⁸ (1.28 × 10⁹) | 4.46% |
| 10⁻⁶ | 24 hours | 6.3 × 10⁵ | 0.67 (3.35 × 10⁶) | 18% |
| 10⁻² | 36 hours | 3.12 × 10⁷ | 2⁸ (1.28 × 10⁹) | 2.4% |
| 10⁻⁶ | 36 hours | 4.15 × 10⁷ | 2⁶ (3.2 × 10⁸) | 12.9% |
| 10⁻² | 48 hours | 1.8 × 10⁷ | 2⁸ (1.28 × 10⁹) | 1.4% |
| 10⁻⁶ | 48 hours | 8.5 × 10⁷ | 2¹⁰ (5.12 × 10⁹) | 1.6% |
| 10⁻² | 72 hours | 5.7 × 10⁶ | 2¹¹ (1.02 × 10¹⁰) | 0.05% |
| 10⁻⁶ | 72 hours | 2.23 × 10⁷ | 2⁹ (2.56 × 10⁹) | 0.87% |

### Quantification of Hemagglutinin Proteins or Viral Proteins

SDS PAGE gel was used to quantify the HA proteins in the viral preparations. ELISA was used for quantification using virus specific serum.

4. Purification

The harvested allantoic fluid was clarified by moderate speed centrifugation (range: 4000-14000 g) first and then separated by sucrose gradient centrifuge; the 35-55% fraction was collected for vaccine preparation.

5. Vaccine Composition and Preparation

Un-treated allantoic fluids or purified viruses were inactivated by 0.1% formalin, and then mixed with mineral oil to make emulsified vaccines.

6. Immunization: Antibody ELISA Data, HI Data

10-day old chicks were immunized with 500 ul of emulsified vaccines via subcutaneous route at the back neck. Sera were collected before challenge.

For the hamaglutination inhibition (HI) assay a virus suspension was incubated with serial (2-fold) dilutions of serum sample pre-treated with cholerafiltrate (obtained from Vibrio cholerae cultures). Subsequently, erythrocytes were added to the dilutions and after incubation the maximum dilution of the agents showing complete inhibition of hamaglutination was defined as the HI antibody titer.

7. Challenge

20 days after immunization the vaccinated chickens were challenged by one H5N1 strain (30 LD₅₀); and the surviving chickens were counted for each group after 5 days post-challenge. The groups vaccinated with viruses from 10⁻² dilution (72 hours) with purification and 10⁻⁶ dilution (36 hours) without purification showed partial protection against H5N1 challenge (5 or 7 survivors respectively), but the group vaccinated with viruses from 10.2 dilution (72 hours) without purification showed no protection against H5N1 challenge (0 survivor).

II. FMDV

1. Viruses

One FMDV strain was selected as the vaccine strain (VS) for its fast growth and stability, two FMDV strains were selected as the challenging strains (CS1 and CS2). VS had a homology of 97.3% over CS1 and a homology of 77.3% over CS2. All viruses were propagated in BHK-21 cells under conventional conditions and procedures.

2. TCID₅₀, PFU and ELISA Results of VS Under Different Dilutions and Harvest Times

The VS working stock was prepared in BHK-21 cells following conventional conditions and procedures and stored at −80° C. The VS working stock was diluted at 10, 100, 1000, 10,000 and 100,000 times and infected BHK-21 cells, where the CPE was recorded at post-infection time of 10, 12, 14, 16, 18, 20 and 22 hours for the calculation of TCID₅₀; each sample was frozen-thaw three times for PFU titers and ELISA experiments. The CPE, PFU and ELISA assays are well known in the art. The results are summarized hereinbelow in Table 2 and Table 3. The TCID₅₀ and PFU values represent the subpopulation of infection viral particles, and the ELISA titer represents the total viral particles or total viral antigens; thus the proportion of the subpopulation of infectious viral particles over the total viral antigens can be expressed as the value of TCID₅₀ or PFU over ELISA readings. It is to be noted that this proportion value is arbitrary, but it is very useful in revealing which virus preparation contains a greater subpopulation of infectious viral particles. If the arbitrary method is consistently used, a predefined value of the proportion of the subpopulation of infectious viral particles can be used to select the conditions for preparing a virus preparation for vaccine production. In addition, the total viral antigens in a virus preparation can be determined by other methods, for example, purifying the total viral antigens and the concentration of the viral antigens can be determined by any known method.

TABLE 2
VS's TCID₅₀ and ELISA titers

| PI | Inoculation of VS working stock with different dilutions | | | | | | | | | | | | | | |
|---|---|---|---|---|---|---|---|---|---|---|---|---|---|---|---|
| Time | 10x | | | 100x | | | 1,000x | | | 10,000 | | | 100.000 | | |
| (h) | A | B | C | A | B | C | A | B | C | A | B | C | A | B | C |
| 10 | 7.6 | 0.31 | 24 | 7 | 0.13 | | 6.6 | 0.04 | 165 | 4.5 | nd | | 4 | nd | |
| 12 | 7.5 | nd | | 73 | nd | | 6.6 | nd | | 6 | nd | | 4.5 | nd | |
| 14 | 6.6 | 0.35 | 18 | 7.6 | 0.24 | 31 | 7.5 | 0.1 | 75 | 6.5 | 0.03 | 210 | 4.5 | nd | |
| 16 | 7.5 | 0.44 | 17 | 8.5 | 0.27 | 31 | 8 | nd | | 6.6 | nd | | 5.3 | nd | |
| 18 | 8 | 0.40 | 20 | 8 | 0.32 | 25 | 8.5 | 0.22 | 38 | 8.3 | 0.11 | 75 | 5.6 | nd | |
| 20 | 7.5 | 0.42 | 17 | 7.5 | 0.35 | 21 | 7.6 | nd | | 7.3 | nd | | 6.6 | nd | |
| 22 | 7.3 | 0.43 | 16 | 17.5 | 0.35 | 21 | 7.5 | 0.28 | 26 | 7.6 | 0.17 | 44 | 6.3 | 0.05 | 126 |

Note:
A represents the TCID₅₀ results (10^{x}/ml);
B represents the ELISA titers (OD450 reading minus the control reading);
C represents the proportion of TCID₅₀ value over ELISA reading.

TABLE 3
VS's PFU and ELISA titers

| PI | Inoculation of VS working stock with different dilutions | | | | | | | | | | | | | | |
|---|---|---|---|---|---|---|---|---|---|---|---|---|---|---|---|
| Time | 10x | | | 100x | | | 1,000x | | | 110,000 | | | 100,000 | | |
| (h) | A | B | C | A | B | C | A | B | C | A | B | C | A | B | C |
| 10 | 3.5 | 0.31 | 11 | 4 | 0.13 | 30 | 2.5 | 0.04 | 62 | 0 | nd | | 0 | nd | |
| 12 | nd | nd | | nd | nd | | nd | nd | | nd | nd | | nd | nd | |
| 14 | 5 | 0.35 | 14 | 3 | 0.24 | 12 | 2 | 0.1 | 20 | 0.35 | 0.03 | 116 | 0 | nd | |
| 16 | nd | 0.44 | | nd | 0.27 | | nd | nd | | nd | nd | | nd | nd | |
| 18 | 6.5 | 0.40 | 16 | 8.5 | 0.32 | 26 | 8.5 | 0.22 | 38 | 1.5 | 0.11 | 13 | 0.05 | nd | |
| 20 | nd | 0.42 | | nd | 0.35 | | nd | nd | | nd | nd | | nd | nd | |
| 22 | 3 | 0.43 | 7 | 3.5 | 0.35 | 10 | 5 | 0.28 | 17 | 4 | 0.17 | 23 | 4.5 | 0.05 | 90 |

Note:
A represents the PFU results (10⁷/ml);
B represents the ELISA titers (OD450 reading minus the control reading);
C represents the proportion of PFU value over ELISA reading.

3. Vaccine Preparation, Immunization and Challenging

From Tables 2 and 3, the two conditions for producing the virus preparations for vaccine production were selected: 10 times dilution and 1000 times dilution; both were incubated for 18 hours before harvest. It was true that some more diluted conditions yielded much higher proportion values, but the amount of the total viral antigens (represented by the ELISA readings) was too low. In practice, the balance between a reasonable yield of total viral antigens and a desired proportion value is needed; if the yield is too low, it becomes uneconomical. The vaccines were produced following conventional procedures. FMDV-negative pigs were immunized twice with a two-week interval. Four weeks after the second immunization, the pigs were challenged with CS1 or CS2 and observed for 18 days. All experiments followed the standard protocols. The results are summarized below.

TABLE 4
Vaccine protection results.

| | Protection after | Protection after |
|---|---|---|
| Vaccine | challenge with CS1 | challenge with CS2 |
| VS 10x dilution | 100% | 60% |
| VS1000x dilution | 100% | 100% |
| Negative control | 0% | 0% |

From Table 4, the increase of the proportion of the subpopulation of infectious viral particles in the vaccines provided better protection against the virus with less homology.

While the present invention has been described with reference to particular embodiments, it will be understood that the embodiments are illustrative and that the invention scope is not so limited. Alternative embodiments of the present invention will become apparent to those having ordinary skill in the art to which the present invention pertains. Such alternate embodiments are considered to be encompassed within the spirit and scope of the present invention. Accordingly, the scope of the present invention is described by the appended claims and is supported by the foregoing description.

What is claimed is:

1. A vaccine comprising:
a viral antigen derived from a virus preparation of a vaccine strain of a virus;
wherein the virus preparation contains a subpopulation of infectious viral particles, and total viral particles (TVPs) or total viral antigens (TVAs); and the infectious viral particles and TVPs or TVAs are over predetermined values.

2. The vaccine of claim 1, wherein when the infectious viral particles are measured as plaque-forming units (PFU) and the TVPS are measured by viral particle numbers, the virus preparation has the following characteristics:
PFU is over 1×10⁷/ml, or 2×10⁷/ml, or 3×10⁷/ml, or 4×10⁷/ml, or 5×10⁷/ml, or 6×10⁷/ml, or 7×10⁷/ml, or 8×10⁷/ml;
TVPs are over 1×10⁸/ml, or 2×10⁸/ml, or 3×10⁸/ml, or 4×10⁸/ml; and
ratio of PFU/TVPs is over 2%, or 3%, or 4%, or 5%, or 6%, or 7%, or 8%, or 9%, or 10%, or 11%, or 12% or 13%.

3. The vaccine of claim 1, wherein when the infectious viral particles are measured as PFU and the TVAs are measured as a numeric value, the virus preparation has the following characteristics:
PFU is over 1×10⁷/ml, or 2×10⁷/ml, or 3×10⁷/ml, or 4×10⁷/ml, or 5×10⁷/ml, or 6×10⁷/ml, or 7×10⁷/ml, or 8×10⁷/ml; and
TVA is about 10-60% of the highest TVA value in a panel of virus preparations containing the virus preparation.

4. The vaccine of claim 1, wherein when the infectious viral particles are measured as TCID₅₀ and the TVAs are measured as a numeric value, the virus preparation has the following characteristics:
TCID50 is over 1×10⁸/ml, or 2×10⁸/ml, or 3×10/ml, or 4×10⁸/ml, or 5×10⁸/ml, or 6×10⁸/ml, or 7×10⁸/ml, or 8×10⁸/ml; and
TVA is about 10-60% of the highest TVA value in a panel of virus preparations containing the virus preparation.

5. The vaccine of claim 1, wherein the virus is able to infect humans and animals.

6. The vaccine of claim 5, wherein the virus is influenza virus, HIV, DENV or FMDV.

7. The vaccine of claim 1, wherein the viral antigen is a surface antigen of the virus.

8. A method for producing a vaccine, said method comprising:
providing a virus preparation of a vaccine strain of a virus; wherein the virus preparation contains a subpopulation of infectious viral particles, and total viral particles (TVPs) or total viral antigens (TVAs), wherein the infectious viral particles and the TVPs or TVAs are over predetermined values;
deriving a viral antigen from the virus preparation; and
mixing the viral antigen with a physiologically acceptable adjuvant to make the vaccine.

9. The method of claim 8, wherein the virus is able to infect humans and animals.

10. The method of claim 9, wherein the virus is influenza virus, HIV, DENV or FMDV.

11. The method of claim 8, wherein the viral antigen is a surface antigen of the virus.

12. The method of claim 8, wherein the predetermined values are determined by:
providing a panel of virus preparations;
measuring the infectious viral particles and TVPs or TVAs of each of the panel of virus preparations;
preparing vaccines with the viral antigens derived from the panel of virus preparations;
immunizing a suitable host with the prepared vaccines;
measuring the cross immune responses of each of the prepared vaccines; and
analyzing the cross immune responses;
wherein the values of the infectious viral particles and TVPs or TVAs from a virus preparation that provides desired cross immune responses are determined as the predetermined values.

13. The method of claim 12, wherein the panel of virus preparations are obtained by:
diluting a virus stock of the vaccine strain into a plurality of dilutions as working stocks;
infecting cells with the working stocks and incubating the infected cells; and
collecting viruses at different incubating time points to obtain the panel of virus preparations.
